(19) United States
(12) Patent Application Publication
(10) Pub. No.: US 2009/0004303 A1
(43) Pub. Date: Jan. 1, 2009

(54) Perfume Compositions

(76) Inventors: Keith Douglas Perring, Kent (GB); John Martin Behan, Kent (GB); David Jonathan Bradshaw, Kent (GB); Briony Care, Kent (GB)

Correspondence Address: MORGAN LEWIS & BOCKIUS LLP, 1111 PENNSYLVANIA AVENUE NW, WASHINGTON, DC, 20004, US

(21) Appl. No.: 11/886,293

(22) PCT Filed: Mar. 17, 2006
(86) PCT No.: PCT/GB2006/000985

§ 371 Date: Jun. 26, 2008

(30) Foreign Application Priority Data
Mar. 18, 2005 (GB) 0505702.1

(51) Int. Cl. A61K 8/97; A61Q 13/00; A61K 36/53; A61K 36/537

(52) U.S. Cl. 424/736; 512/5; 424/745; 424/746; 424/725

(57) ABSTRACT

Perfume ingredients and essential oils in synergistic combinations improve well-being by down-regulating arousal. This could lead to enhanced quality of life and reduced stress. The perfume compositions of the invention comprise one or more materials from each of the following groups: Group A: the essential oils lavender, lavandin, bergamot, chamomile, clary sage; Group B: 2-phenoxyethanol, 1-(2,3,8,8-tetramethyl-1,2,3,4,5,6,7,8-octahydronaphthalen-2-yl)ethanone, benzophenone, cyclopentadecanolide, alpha-ionone, beta-ionone, para-methylacetophenone, [4-isopropylcyclohexyl]methanol. The ratio of the weight percentage based on the composition of group A materials to Group B materials lies within the range 1:9 to 9:1.

### FIELD OF THE INVENTION

This invention relates to perfume compositions (also referred to as fragrance compositions). A perfume composition is a mixture of perfume ingredients, if desired mixed with or dissolved in a suitable solvent or solvents and/or mixed with a solid substrate. Perfume ingredients are well known to those skilled in the art, and include those mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials—1991”, Allured Publishing Co. Wheaton, Ill. USA. Perfume ingredients may include natural products such as extracts, essential oils, absolutes, resinoids, resins, concretes etc., and also synthetic basic substances such as hydrocarbons, alcohols, aldehydes, ketones, ethers, acids, esters, acetals, ketals, nitrites, etc., including saturated and unsaturated compounds, aliphatic, arbocyclic and heterocyclic compounds. The invention is particularly concerned with perfume compositions that have the ability to down-regulate arousal and promote stress relief through olfactory stimulation.

### BACKGROUND TO THE INVENTION

Much has been written in recent years concerning the adverse effects of stress. It has been established that stress may be caused by upheavals such as divorce, loss of a job, change of location and similar events, and often may precede illness. Headey and Wearing found that negative life events led to decreases in subjective well-being (Headey B and Wearing A, 1989, Journal of Personality and Social Psychology, 57, 731).

Numerous books have been written on ways to relax and relieve stress and anxiety. Methods of relaxation include exercise systems and bio-feedback systems that have been developed to aid both physical and mental relaxation. Such systems are employed normally in conjunction with physical devices that are designed to stimulate the senses, particularly the sense of sight and sound, and are generally intended to provide an environment that shuts out external interferences.

Some devices go as far as to provide an enclosure wherein the senses may be bombarded for the purpose of inducing a state of relaxation. Other devices have been employed which are less elaborate but which may involve a headset for transmitting sound and a means for providing a visual display in conjunction with the sound.

An example of a system for promoting relaxation where stimulation of the olfactory senses occurs is described in U.S. Pat. No. 4,640,266. However, the user must be within an enclosure and he/she is also subjected to visual and auditory stimulation.

Popular methods for inducing relaxation include “aromatherapy”.

In general, aromatherapy is a term used to describe the use of essential oils, more particularly the fragrance or aroma from essential oils, to promote natural treatment of various bodily and psychic dysfunctions. The expansion of modern aromatherapy began in Europe in 1964 with the publication of the book “aromatherapie”, which has been translated in the English under the name “The Art of Aromatherapy Healing”, Arts Press, Rochester, Vt., 1982.

Essential oils are highly concentrated extracts from certain plants and are characterized by being volatile oily substances. They may be produced by a number of different processes including solvent extraction, pressing and distillation.

In general, essential oils are low molecular weight terpene-based or shikimate-based derivatives that are substituted with various functional groups, such as ketone, aldehyde, ester, alcohol and the like. These groups give the various essential oils their characteristic properties. Essential oils may be produced from practically all parts of a plant, including the blossom, seeds, wood and leaves.

In accordance with the theories of aromatherapy, certain essential oils or blends of essential oils, are known to affect different parts of the body. Thus, certain essential oils, and blends thereof, are known to be effective in the relief of stress and in the inducement of relaxation.

Additionally, it is well known that fragrances can induce a state of mind such as a pleasant sensation, as is well evidenced by the art of perfumery.

However, the limited number of available “relaxing” essential oils restricts the ability to produce diverse fragrances that induce relaxation. There exists a need for fragrance compositions or accords that may be used as desired in the fragrance industry to down-regulate arousal and reduce stress.

Reactivity to stress is insidious because even though it may not directly incapacitate, researchers have discovered that stress decreases productivity, and eventually may lead to illness. The first effect of stress is to imbalance the individual who will need energy to react to a change in his environment. This is followed by a period of adaptive response which sees the body's resources used to counter the stressor. However, if the stress persists exhaustion will follow. This reactivity to stress can be brought about by the repetitive or chronic strains of everyday life. Stress can alter the ability of the organism to respond to external threats through impaired immunity. For a discussion see Glaser et al (eds.), “Handbook of Human Stress and Immunity”, Academic Press Inc., 1994.

The reaction of the neuroendocrine system to stress can be seen in levels of glucocorticoid hormones, in particular cortisol. Raised levels of cortisol can result from increased stress.

Classical techniques used to promote relaxation and control stress include bio-feedback, meditation and the use of chemical agents.

Bio-feedback includes pulse transit time, electromyogram activity and skin resistance bio-feedback.

Meditation generally involves techniques that are intended to lower blood pressure by promoting physical and mental relaxation. Specific examples of such techniques are disclosed in U.S. Pat. No. 4,670,463.

A review of the use of bio-feedback and meditation techniques for the treatment of hypertension can be found in: Surwit et al, “Behavioral Approaches to Cardiovascular Disease”, Behavioral Medicine Series, Academic Press, 1982, 139-156.

Chemical agents to modify the effects of stress, tension, anxiety and dysphoria have been sought throughout recorded history. Probably, the oldest drug for this use is ethanol. In the last century, bromide salts and the barbiturates were introduced. Barbiturates continued to be the dominant anti-anxiety agents until the 1950's, when propandiol carbamates (MEPROBAMATE®), and congeners were introduced. The side effects of the barbiturates and the propandiol carbamates, in particular, the physical dependence caused by these drugs and the severe acute intoxication on over dosage, encouraged the search for more specific anti-anxiety drugs. Today, the benzodiazepines series of drugs, chlordiazepoxide and diazepam in particular, are the major drugs used for treatment of anxiety and stress. A review of the drug treatment of stress and anxiety can be found in: Goodman and Gilman, “Pharmacological Basis of Therapeutics”, 6th ed., MacMillan Publishing Co., New York, N.Y., (1980), pages 436-446.

Aromatherapy is a method for reducing physiological and/or subjective reactivity to stress. In fact, it is currently a burgeoning industry that has led to the commercial success of a vast variety of aromatherapy oils, soaps, perfumes, massage treatments and the like. The term “Aromatherapy” is intended herein to mean the use of essential oils derived from plants for the treatment of health problems.

Aromatherapy techniques are reviewed in: (1) J. Valnet, “The Practice of Aromatherapy”, Destiny Books (Division of Inner Traditions International, Ltd.), New York, N.Y., 1982; (2) R. Tisserand, “The Art of Aromatherapy”, Destiny Books (Division of Inner Traditions International, Ltd.), New York, N.Y., 1983; and (3) A. Leung, “Encyclopedia of Common Natural Ingredients”, J. Wiley & Sons Publishing Co., New York, N.Y., 1980.

Although the use of essential oils is well known for the promotion of relaxation, the inventors of the subject invention have discovered that by using certain essential oils that are common to the fields of perfumery and aromatherapy, in combination with certain non-essential oil fragrance ingredients, fragrance compositions and accords may be produced that reduce levels of arousal in subjects, particularly human subjects, thus leading to relaxation (i.e. down-regulated arousal) and so are useful for reducing stress.

### SUMMARY OF THE INVENTION

In one aspect the present invention provides a perfume composition, suitable for use in fragrances that down-regulate arousal, comprising one or more materials from each of the following groups:

Group A: the essential oils lavender, lavandin, bergamot, chamomile, clary sage;
Group B: 2-phenoxyethanol, 1-(2,3,8,8-tetramethyl-1,2,3,4,5,6,7,8-octahydronaphthalen-2-yl)ethanone, benzophenone, cyclopentadecanolide, alpha-ionone, beta-ionone, para methylacetophenone, [4 isopropylcyclohexyl]methanol (also known by the semi-trivial name para-menthane-7-ol);
and wherein the ratio of the weight percentage based on the composition of Group A materials to Group B materials lies in the range 1:9 to 9:1.

Inhalation of the composition by a subject can down-regulate arousal, thus inducing a more relaxed state and so promoting the reduction of stress.

The dose levels of the essential oils and non-essential oil fragrance ingredients in the perfume compositions of this invention may differ from those normally employed in either perfumery or aromatherapy. But by using selected doses of such essential oils and non-essential oil fragrance ingredients, individuals can experience some degree of stress reduction.

The invention is particularly applicable to perfume compositions in the form of fragrance accords. The term ‘fragrance accord’ is used to denote fragrance compositions comprising a harmonious combination of a small number of notes; accords may be regarded as building blocks to complex perfumes (see David Williams “Perfumery of Yesterday”, Micelle Press, 2004, page 99).

Thus although the essential oils employed in the practice of this invention are known in the art and are known to exhibit physiologic activity, there is no suggestion in the prior art that the combination of these essential oils with non-essential oil materials could down-regulate arousal, relieve stress and possibly enhance relaxation.

The essential oils of this invention are selected from the group consisting of lavender, lavendin, bergamot, chamomile, clary sage and mixtures thereof. These essential oils are common perfumery ingredients as described in Arctander, “Perfume and Flavors Materials of Natural Origin”, published by the author in 1960.

The non-essential oil fragrance ingredients of this invention are selected from the group consisting of 2-phenoxyethanol, 1-(2,3,8,8-tetramethyl-1,2,3,4,5,6,7,8-octahydronaphthalen-2-yl)ethanone (e.g. in the form of Iso Ambois™—Iso Ambois is a Trade Mark), benzophenone, cyclopentadecanolide, alpha-ionone, beta-ionone, para-methylacetophenone, [4-isopropylcyclohexyl]methanol (also known by the semi-trivial name para-menthane-7-ol) and mixtures thereof.

Compositions in which at least 75% of the group A essential oils present comprise either lavender or bergamot are preferred.

It is preferred that at least two group A materials are present. It is advantageous for at least two group B materials to be present, even more preferably at least three.

The weight ratio of group A to group B materials may lie anywhere in the range 9:1 to 1:9 but is preferably 3:1 to 1:3, more preferably 2:1 to 1:2.

The term “stress” hereinafter refers to an event or experience in the life of an individual that has specific physiologic and/or subjective consequences that disturb the equilibrium of the individual (Glock, C. Y. & Leonard, H. L., Journal of Chronic Diseases, 1956, 5, 179).

Reactivity to stress varies with the individual. Some individuals thrive on stress whereas in other individuals, the same stress drives them towards sickness (Executive Fitness Newsletter, Rodale Press Inc., Vol. 15, No. 17 [1984]). However, in general stress has a deleterious effect on health, and can be debilitating to sufferers, and reduces quality of life of chronic sufferers.

The invention can promote relaxation and the reduction of stress in a subject, particularly a human subject. Stress relief is achieved through the stimulation of the sense of smell of the subject, through the inhalation by the subject of an effective amount of at least one perfume composition in accordance with the invention, particularly in an aesthetically pleasing form, such as, for example, cologne, a perfumant or an air freshener.

The fragrance composition or accord may be administered alone or as part of a consumer product.

Consumer products that may comprise fragrance compositions or accords according to this invention include, for example, room fresheners or room deodorants; clothes deodorants; fabric softeners; household cleansers; toilet bowl cleaners; cosmetic products such as antiperspirant and underarm deodorants, general body deodorants, hair care products such as hair sprays, conditioners, rinses, shampoos; foot care products; colognes, after shaves and body lotions; soaps and synthetic detergents; and odour control products.

These products may take a variety of forms including powders, bars, sticks, tablets, mousses, gels, liquids, sprays, and also fabric conditioning sheets.

The invention thus covers a consumer product, particularly a personal care product, comprising a perfume composition in accordance with the invention, optionally containing additional perfumery materials. The product suitably comprises at least 0.01% by weight, preferably at least 0.1% by weight, of the perfume composition.

An example of the use of fragrance accords according to this invention to down regulate arousal, is their inclusion in fragrance compositions for use in air freshening products at a level that will provide from about 1 to about 125 micrograms per liter of fragrance the air of a room.

The results obtained through practice of this invention are comparable to results obtainable from meditation and bio-feedback. However, unlike meditation or bio-feedback, no training period is required in the use of the relaxation promoting and stress relieving substances of our invention.

Unlike chemical agents for the relief of stress, the effect of the fragrance accords employed in the practice of our invention is prophylactic in nature.

Effective dosage levels of the fragrance compositions and accords of the present invention will depend upon the method of delivery to the olfactory system. So for example when dispensed in household products such as toilet bowl fresheners dosage levels of up to 5% or even 10% by weight may be employed. However in many personal products much lower levels may be sufficient. For example, in a face cream levels of 0.01% may suffice, more typically 0.05% or even 0.1%. Directions for quantity to use and frequency of use, as well as variations in the formulation, e.g., summer and winter formulations, may be employed to assure that effective levels of the fragrances may be administered. For the purpose of this invention, the term “cologne”, as exemplified hereinafter, means a fragrance incorporated in an alcoholic or hydroalcoholic solution. The fragrance can vary between 1 to 25% and the balance of the formulation is comprised of alcohol or a mixture of water and alcohol. The water:alcohol weight ratio can vary from 50:50 to 0:100. Examples of alcohols typically used in these products are SDA 39-C and SDA-40, either 190 “proof” or anhydrous (See “Ethyl Alcohol Handbook”, 5th Edition, Published by National Distillers and Chemical Co.). The cologne can also contain solubilizing agents, emollients, humectants, thickening agents, bacteriostats or other cosmetically used ingredients.

Perfumery materials, that are compatible with the fragrances comprising fragrance accords of this invention, may be employed in fragrancing perfumed articles. Such perfumed articles may include fabric softener compositions, dryer-added fabric softener articles (such as BOUNCE® (registered trade mark of the Procter & Gamble Company)), cosmetic powders, talc, solid or liquid anionic, cationic, nonionic or zwitterionic detergents and perfumed polymers as well as deodorant sticks, hair preparations and bar soaps. Thus, a great number of state-of-the-art perfume compositions and perfumed articles are available for use as the non-active carrier composition and articles within which the fragrances comprising fragrance accords may be incorporated for practice of this invention.

Thus, the fragrances that down-regulate arousal may be used alone or taken together with carrier perfume compositions alone or through carrier perfumed articles. Many well known consumer products may be the carrier such as solid or liquid anionic, cationic, nonionic or zwitterionic detergents bar soaps, space odorants and deodorants; colognes, toilet waters, hair preparations, such as lacquers, brilliantines, and pomades; cosmetic preparations, such as creams, deodorants, hand lotions and sun screens; and powders, such as talc, dusting powders, face powders and the like. It is interesting to note that when a fragrance that down-regulates arousal according to this invention is used in a deodorant stick or deodorant bar a two-fold effect takes place:

(i) the deodorant stick itself acts as a “deodorant” in the axillary area of the human being; and
(ii) the fragrance is administered by inhalation to promote a reduction in stress.

The term “perfumed article” also includes solid-form polymers, such as polyethylene, polypropylene and other polymers that contain pores. Such perfumed polymers can be produced according to any technique well known to one having ordinary skill in the art.

Subjective well-being can be measured by self-reports, which may consist of single item or multiple item questionnaires. These may be qualitative or quantitative. Subjects can reflect their level of happiness. For example, see Sandvik E., Diener E. and Seidlitz L., 1993, Journal of Personality, 61, 317, and the methods referenced therein.

Other methods for measuring changes in emotional, mood, and well-being may employ electroencephalography (EEG) to monitor changes in brain-wave activity. See, for example, PCT Application WO 02/49600 in which alpha-wave activity is shown to be altered by relaxing fragrance ingredients alone.

The invention also includes within its scope a method of causing the down-regulation of arousal in a subject, comprising the inhalation by the subject of a perfume composition or a consumer product in accordance with the invention.

In a further aspect, the invention covers use of a perfume composition or consumer product in accordance with the invention for the purpose of down-regulating arousal.

The following Examples further describe and illustrate preferred embodiments within the scope of the present invention. The Examples are given solely for the purpose of illustration and are not to be construed as limitations of the present invention as many variations thereof are possible without departing from its spirit and scope. All percentages, ratios, and parts herein, are by weight and are approximations, unless otherwise stated.

The group A materials are preferably selected from lavender, bergamot, chamomile and clary sage, and the group B materials are preferably selected from the materials listed above excluding beta-ionone.

### EXAMPLES

### 1. Fragrance Accord Compositions

Table 1 illustrates accords within the scope of the invention, Table 2 lists some comparative examples.

TABLE 1
Compositions of the invention

| | | ACCORD | | | | | | |
|---|---|---|---|---|---|---|---|---|
| COMPONENT | GROUP | 1 | 2 | 3 | 4 | 5 | 6 | 7 |
| BENZOPHENONE | B | 5 | 0 | 0 | 0 | 0 | 0 | 0 |
| BERGAMOT | A | 40 | 0 | 0 | 75 | 60 | 0 | 7 |
| CHAMOMILE | A | 0 | 6.5 | 0 | 0 | 0 | 0 | 0 |
| CLARY SAGE | A | 0 | 0 | 2 | 0 | 0 | 0 | 0 |
| CYCLOPENTADECANOLIDE | B | 50 | 65 | 0 | 0 | 8 | 0 | 0 |
| IONONE ALPHA | B | 0 | 0 | 13 | 0 | 0 | 0 | 0 |
| IONONE BETA | B | 0 | 0 | 0 | 0 | 0 | 0 | 0 |
| ISO AMBOIS ™ | B | 0 | 0 | 67 | 0 | 20 | 15 | 10 |
| LAVENDER | A | 0 | 22 | 18 | 10 | 0 | 7 | 0 |
| LINALOL | — | 0 | 0 | 0 | 0 | 0 | 20 | 10 |
| LINALYL ACETATE | — | 0 | 0 | 0 | 0 | 0 | 13 | 23 |
| p-MENTHANE-7-OL | B | 0 | 0 | 0 | 15 | 2 | 0 | 0 |
| p-METHYL ACETOPHENONE | B | 5 | 6.5 | 0 | 0 | 0 | 0 | 0 |
| METHYL | — | 0 | 0 | 0 | 0 | 0 | 15 | 25 |
| DIHYDROJASMONATE | | | | | | | | |
| 2-PHENOXYETHANOL | B | 0 | 0 | 0 | 0 | 10 | 0 | 25 |
| TONALID ™ | — | 0 | 0 | 0 | 0 | 0 | 30 | 0 |
| total % | | 100 | 100 | 100 | 100 | 100 | 100 | 100 |
| Sum Group A | | 40 | 28.5 | 20 | 85 | 60 | 7 | 7 |
| Sum Group B | | 60 | 71.5 | 80 | 15 | 40 | 15 | 35 |
| Ratio SumA/SumB | | 0.67 | 0.40 | 0.25 | 5.67 | 1.50 | 0.47 | 0.20 |

TABLE 2
Fragrance accord compositions: comparative examples

| COMPONENT | GROUP | C1 | C2 | C3 | C4 |
|---|---|---|---|---|---|
| BENZOPHENONE | B | 0 | 0 | 0 | 0 |
| BERGAMOT | A | 0 | 10 | 0 | 80 |
| CHAMOMILE | A | 0 | 0 | 5 | 0 |
| CLARY SAGE | A | 0 | 0 | 0 | 0 |
| CYCLOPENTADECANOLIDE | B | 0 | 0 | 0 | 0 |
| IONONE ALPHA | B | 0 | 0 | 0 | 0 |
| IONONE BETA | B | 0 | 0 | 0 | 0 |
| ISO AMBOIS ™ | B | 0 | 0 | 0 | 0 |
| LAVENDER | A | 10 | 0 | 45 | 0 |
| LINALOL | — | 60 | 30 | 0 | 0 |
| LINALYL ACETATE | — | 30 | 60 | 0 | 0 |
| p-MENTHANE-7-OL | B | 0 | 0 | 0 | 0 |
| p-METHYL ACETOPHENONE | B | 0 | 0 | 0 | 0 |
| METHYL | — | 0 | 0 | 0 | 10 |
| DIHYDROJASMONATE | | | | | |
| 2-PHENOXYETHANOL | B | 0 | 0 | 0 | 0 |
| TONALID ™ | — | 0 | 0 | 50 | 10 |
| total % | | 100 | 100 | 100 | 100 |
| Sum Group A | | 10 | 10 | 50 | 80 |
| Sum Group B | | 0 | 0 | 0 | 0 |
| Ratio SumA/SumB | | — | — | — | — |

### 2. Measurement of Salivary Cortisol as an Indicator of Stress Relief

Salivary cortisol was measured using a competitive Enzyme Linked Immunosorbent Assay (ELISA) test kit (DRG Instruments GmbH, Marburg, Germany). Briefly, saliva samples were collected by expectoration. 100 μl aliquots of these samples and of cortisol standard solutions were dispensed into the appropriate wells of a 96-well microtitre plate. 200 μl of horseradish peroxidase-cortisol conjugate solution was then added to each well, shaken for 10 seconds, and then incubated for 60 minutes at 21° C. The contents of the microtitre plate wells were then shaken out briskly, and the plate washed three times with wash solution supplied with the kit. 200 μl of substrate (tetramethyl benzidine/H₂0₂) was then added and the plate incubated at 21° C. for 30 minutes. The reaction was stopped by addition of 100 μl of 0.5M H₂SO₄. The absorbance of each well was then measured at 450 nm in a microtitre plate reader (Model MRX, Dynatech Laboratories, Billinghurst, Sussex, UK). Cortisol values for each saliva sample were then calculated by comparison with a standard curve of absorbance against cortisol concentration of the standard samples.

The above protocol was applied in a stress induction study based on the Social Stress Test (Kirschbaum, Pirke and Hellhammer 1993, Neuropsychobiology, 28, 76-81). Stress levels were measured on a control group (unstressed), and on two test groups A and B subjected to identical stress, but only group B was exposed to perfume compositions of the invention. Typical cortisol levels in the control group were in the range 1 ng/ml to 2 ng/ml. Cortisol levels for group A were typically above 3 ng/ml, whilst those for group B were typically lower than 3, indicating a stress reduction.

### 3. Measurement of Salivary IgA as an Indicator of Stress Relief

Salivary IgA was assayed by an immunoturbidometric assay. Samples of saliva were incubated with rabbit anti-human IgA antibody (DAKO, Cat No Q0322), in the presence of polyethylene glycol (PEG). Reagents were added in the following order to appropriate wells of a microtitre plate: 120 microlitre 2% PEG, 10 μl sample or standard, 90 μl PBS and 30 μl rabbit anti-IgA. At the same time a standard range of calibrator solutions (DAKO, Cat No X0908) of 25, 50 and 100 mg/dl were also prepared. The microtitre plate was shaken gently to mix the reagents and allowed to stand for 30 minutes at room temperature. The optical density of the precipitate was read following an automatic shaking step at 340 nm using a Model MRX plate reader (Dynatech Laboratories, Billingshurst, Sussex, UK).

Salivary IgA concentrations for a control (“non-stressed”) subject were in the range ca. 20-50 mg/dl (mean ca. 30 mg/dl). A series of subjects stressed by completion of the Trier stress test (Kirschbaum et al., 1993) had markedly lower levels of IgA (means lower than 20 mg/dl). Subjects exposed to perfume compositions of the invention exhibited lower levels of stress activation.

1. A perfume composition comprising one or more materials from each of the following groups:
Group A: the essential oils lavender, lavandin, bergamot, chamomile, clary sage;
Group B: 2-phenoxyethanol, 1-(2,3,8,8-tetramethyl-1,2,3,4,5,6,7,8-octahydronaphthalen-2-yl)ethanone, benzophenone, cyclopentadecanolide, alpha-ionone, beta-ionone, para-methylacetophenone, [4-isopropylcyclohexyl]methanol;
and wherein the ratio of the weight percentage of Group A materials to Group B materials based on the total weight of the composition lies within the range 1:9 to 9:1.

2. A composition according to claim 1, wherein the weight ratio of Group A materials to Group B materials lies within the range of 3:1 to 1:3.

3. A composition according to claim 2, wherein the weight ratio of Group A materials to Group B materials lies within the range 2:1 to 1:2.

4. A composition according to claim 3, wherein at least two materials from group A are present.

5. A composition according to claim 1, wherein at least 75% of the Group A materials comprises lavender.

6. A composition according to claim 1, wherein at least 75% of the Group A materials comprises bergamot.

7. A composition according to claim 1, comprising at least two materials from Group B.

8. A composition according to claim 1, in the form of a fragrance accord.

9. A consumer product for the down-regulation of arousal, comprising a fragrance composition in accordance with claim 1, optionally containing additional perfumery materials.

10. A product according to claim 9, comprising at least 0.01% by weight, preferably at least 0.1% by weight, of the perfume composition.

11. A product according to claim 9 or 10, in the form of a personal care product.

12. A method of causing the down-regulation of arousal in a subject, comprising the inhalation by the subject of a perfume composition or a consumer product in accordance with claim 1.

13. (canceled)
